(19) United States
(12) Patent Application Publication
(10) Pub. No.: US 2026/0053484 A1
(43) Pub. Date: Feb. 26, 2026

(54) System and Method for Holding Open a Surgical Incision

(71) Applicant: OrthoTract Innovations, LLC, Greenville, SC (US)

(72) Inventors: Jacqueline Jo Hanna, Washington, DC (US); Kaila Nicole Wallace, Fort Mill, SC (US); Gretchen Jo Finley, Greenville, SC (US); Steven Blake Durden, Schertz, TX (US); Michael J. Stokes, Cincinnati, OH (US)

(73) Assignee: OrthoTract Innovations, LLC, Greenville, SC (US)

(21) Appl. No.: 19/305,232

(22) Filed: Aug. 20, 2025

Related U.S. Application Data
(60) Provisional application No. 63/685,186, filed on Aug. 20, 2024.

(51) Int. Cl. A61B 17/02; A61B 90/50

(52) U.S. Cl. CPC A61B 17/02; A61B 90/50

(57) ABSTRACT

A system or method can hold open a surgical incision or other cut or tissue opening on one side of an appendage via actuation initiated or occurring on an opposite side of the appendage. A device, such as a retractor, can extend at least partially about the appendage and can convey, transmit, or carry motion and/or force between the two sides of the appendage. For example, a surgeon or other user can squeeze a retractor handle that is disposed on the opposite side of the appendage. Responsive to the squeeze, the retractor can apply opening motion and/or force to the incision, so as to hold the incision open during surgery or some other procedure.

### CROSS-REFERENCE TO RELATED APPLICATIONS

This application claims priority to U.S. Provisional Patent Application No. 63/685,186 filed Aug. 20, 2024 in the name of Jacqueline Jo Hanna, Kaila Nicole Wallace, Gretchen Jo Finley, Steven Blake Durden, and Michael J. Stokes and entitled “System and Method for Holding Open a Surgical Incision,” the entire contents of which are hereby incorporated herein by reference. Thus, the entire contents of U.S. Provisional Patent Application No. 63/685,186 are incorporated herein by reference.

### TECHNICAL FIELD

Embodiments of the technology relate generally to holding open a surgical incision, and more particularly to holding open a surgical incision on one side of an appendage via actuation initiated or occurring on an opposite side of the appendage, for example using a retractor that extends at least partially about the appendage.

### BACKGROUND

In many respects, conventional technologies underserve holding surgical incisions open. For instance, need exists for holding open a surgical incision on an appendage in a manner that provides a sufficient or desired level of surgical access to tissue exposed by the surgical incision and remain in place during the procedure.

Further need exists for a capability to hold open a surgical incision so that the surgical incision remains sufficiently or desirably open throughout a surgical procedure or another medical procedure, for instance to avoid inadvertent closure or partial closure of the surgical incision, thereby dislodging an instrument or otherwise causing an interruption to the procedure.

Further need exists for a capability to open a surgical incision efficiently and to hold open the surgical incision efficiently and consistently.

Further need exists for a capability to reduce the concentrated pressure placed on tissue during surgery, for example to minimize localized trauma, reduce the risk of ischemia or tissue necrosis, and improve overall patient safety while maintaining effective exposure of the surgical site.

Further need exists for a capability to maintain the appendage in a desired rotational orientation during surgery, for example so the surgical incision is vertically oriented or faces a surgeon, and further avoiding inadvertent dislodgement during the surgical procedure. Thus, need exists for maintaining a surgical incision in a desired position.

The aforementioned needs are representative rather than exhaustive. A technology addressing one or more of the needs discussed above, or some related deficiency in the art, would benefit medicine. As will be appreciated by those having skill in the art, the disclosure provided herein includes written description containing clear, exact terms to enable carrying out embodiments meeting the foregoing needs.

### SUMMARY

A system or method can hold open a surgical incision on one side of an appendage via actuation initiated or occurring on an opposite side of the appendage.

In some aspects of the disclosure, a method can be practiced for holding open a surgical incision on a volar side of a forearm. In accordance with the method, a handle of a retractor can be disposed on a dorsal side of a forearm, entrapping the retractor onto the surgical site to help avoid dislodgement. A user can exert compressive force on the handle. The retractor can comprise a pair of members and a pivot joint that are operably coupled to the handle. The pair of members and the pivot joint can convert the compressive force into expansive force between two prongs of the retractor that are disposed in the surgical incision on the volar side of the forearm. The two prongs can hold open the surgical incision.

In some subaspects of the disclosure, the two prongs disposed in the surgical incision can comprise: a first prong disposed in the surgical incision and applying a first force to a first side of the surgical incision; and a second prong disposed in the surgical incision applying a second force to a second side of the surgical incision. The first force and the second force can oppose one another. The first force and the second force can be substantially parallel or substantially collinear.

In some subaspects of the disclosure, the two prongs disposed in the surgical incision can comprise: a first prong disposed in the surgical incision against a first side of the surgical incision; and a second prong disposed in the surgical incision against a second side of the surgical incision. The first prong can apply to the first side of the surgical incision a first force comprising a first magnitude and a first direction. The second prong can apply to the second side of the surgical incision a second force comprising a second magnitude and a second direction. The first and second magnitudes can be substantially equal. The second direction can be substantially opposite the first direction.

In some subaspects of the disclosure, the method further comprises the handle or other member of the retractor pressing against a surface that supports the forearm during surgery to impede pronation of the forearm. The surface can comprise an surgical table, for example.

In some subaspects of the disclosure, the handle of the retractor can be biased laterally relative to the forearm.

In some aspects of the disclosure, a retractor can comprise a first member, a second member, a pivot joint, and a locking mechanism. The first member can comprise a first finger ring; a first portion of the pivot joint; a first handle portion extending between the first finger ring and the pivot joint; a first prong; and a first shank extending between the pivot joint and the first prong. The second member can comprise: a second finger ring; a second portion of the pivot joint; a second handle portion extending between the second finger ring and the pivot joint; a second prong; and a second shank extending between the pivot joint and the second prong. The locking mechanism can extend between the first and second handle portions. The first shank can extend lengthwise away from the pivot joint along a first side of an axis and then extend in a first rotational direction partially about the axis to a second side of the axis that opposes the first side of the axis. The first shank can orient the first prong on the second side of the axis with the first prong extending towards the axis. The second shank can extend lengthwise away from the pivot joint along the first side of the axis and then extend in a second rotational direction partially about the axis to the second side of the axis. The first shank can be longer than the second shank so as to dispose the finger rings away from an axis of an incision or other cut or tissue opening. The first and second rotational directions can oppose one another. The second shank can orient the second prong on the second side of the axis with the second prong extending towards the axis.

In some subaspects of the disclosure, the first prong can curve in the first rotational direction as the first prong extends towards the axis, the second prong can curve in the second rotational direction as the second prong extends towards the axis, the axis can comprise an axis of an appendage, and the retractor can be configured to hold open a surgical incision on the appendage.

In some subaspects of the disclosure, the first and second finger rings can form a handle that is laterally offset relative to the axis.

In some subaspects of the disclosure, at least one of the first shank and the second shank can comprise a straight section that is extendable, or alternatively a first shank can be made longer or shorter than the second shank.

In some subaspects of the disclosure, the first prong can be rotatable about a second axis that is substantially perpendicular to the axis and the second prong can be rotatable about a third axis that is substantially perpendicular to the axis.

In some subaspects of the disclosure, the first shank can comprise a first lock for fixing the first prong in a first rotational and/or elevation position about the second axis, and the second shank can comprise a second lock for fixing the second prong in a second rotational and/or elevation position about the third axis.

In some subaspects of the disclosure, the first shank can comprise a first section that extends in the first rotational direction partially about the axis to the second side of the axis, the second shank can comprise a second section that extends in the second rotational direction partially about the axis to the second side of the axis, a first portion of the first section can be adjustably extendable for setting first separation between the first prong and the axis, and a second portion of the second section can be adjustably extendable for setting second separation between the second prong and the axis.

In some subaspects of the disclosure, a reference plane can extend between the first prong and the second prong and can comprise the axis, a first portion of the first shank can be disposed adjacent the first prong and can be compliant to the reference plane, and a second portion of the second shank can be disposed adjacent the second prong and can be compliant to the reference plane.

In some aspects of the disclosures a method can be practiced for holding open a surgical incision or other cut or opening on a first side of an appendage. The method can comprise providing a self-retaining retractor. The self-retaining retractor can comprise: a handle comprising a pair of finger rings; a pair of prongs disposed opposite the handle; a joint disposed between the handle and the pair of prongs; and a pair of members that extend between handle and the pair of prongs and that meet at the joint. The method can further comprise the self-retaining retractor holding open the surgical incision, with the handle disposed adjacent a second side of the appendage that is opposite the first side of the appendage.

In some subaspects of the disclosure, holding open the surgical incision can comprise the pair of prongs extending into and holding open the surgical incision while each prong in the pair of prongs is free to rotate about a respective axis that extends vertically.

In some subaspects, the method can further comprise the self-retaining retractor locking each prong in the pair of prongs to a user-selected angular position about the respective axis that extends vertically.

In some subaspects of the disclosure, the method can further comprise the self-retaining retractor locking each prong in the pair of prongs to a respective user-selected extension length that sets a respective depth of extension into the surgical incision.

In some subaspects of the disclosure, the method can further comprise the self-retaining retractor impeding rotation of the appendage with the handle disposed between the second side of the appendage and a surface that supports the appendage during surgery.

In some subaspects of the disclosure, the handle can be biased laterally relative to the appendage and can be disposed between the second side of the appendage and a surface supporting the appendage. And, the method can further comprise impeding rotation of the appendage via interaction between the biased handle and the surface.

In some subaspects of the disclosure, the handle can be disposed adjacent the second surface of the appendage and can be biased to a lateral side of the appendage. And, the method can further comprise the self-retaining retractor holding open the surgical incision on the first side of the appendage with the pair of prongs extending into the surgical incision.

In some subaspects of the disclosure, the pair of prongs can comprise a first prong and a second prong. And, the method can further comprise the self-retaining retractor lengthening the first prong relative to the second prong responsive to user input.

The foregoing discussion about holding open surgical incisions is for illustrative purposes only. Various aspects of the present disclosure may be more clearly understood and appreciated from a review of the following text and by reference to the associated drawings and the claims that follow. The Figures and specification support each aspect and subaspect of the disclosure that this Summary identifies. This Summary does not intend to be exhaustive, nor does it intend to enumerate each and every aspect of the disclosure. Other aspects, systems, methods, features, advantages, and objects of the present disclosure will become apparent to those with skill in the art upon examination of the following drawings and text. It is intended that all such aspects, systems, methods, features, advantages, and objects are to be included within this description and covered by this paper and by the appended claims.

### BRIEF DESCRIPTION OF THE FIGURES

FIG. 1 is an illustration of a representative application or usage environment of a system for holding open a surgical incision, whereby a self-retaining retractor holds open the surgical incision in accordance with some example embodiments of the disclosure.

FIGS. 2A, 2B, and 2C (collectively FIG. 2) are illustrations of a self-retaining retractor in accordance with some example embodiments of the disclosure.

FIGS. 3A, 3B, 3C, 3D, 3E, 3F, and 3G (collectively FIG. 3) are illustrations of a self-retaining retractor in combination with a cylinder that represents an appendage comprising a surgical incision that the self-retaining retractor is configured to hold open in accordance with some example embodiments of the disclosure.

FIGS. 4A, 4B, 4C, and 4D (collectively FIG. 4) are illustrations of a self-retaining retractor that comprises different shank lengths in combination with a cylinder that represents an appendage comprising a surgical incision that the self-retaining retractor is configured to hold open in accordance with some example embodiments of the disclosure.

FIGS. 5A, 5B and 5C (collectively FIG. 5) are illustrations of a self-retaining retractor that comprises adjustable shank lengths in accordance with some example embodiments of the disclosure.

FIG. 6 is an illustration of a self-retaining retractor that comprises a pair of shanks, respective portions of which are compliant, in accordance with some example embodiments of the disclosure.

FIG. 7 is an illustration of a self-retaining retractor that comprises a pair of shanks, respective portions of which are extendable and rotatable, each with a mechanism for setting extension and rotation and that comprises a knob in accordance with some example embodiments of the disclosure.

FIG. 8 is an illustration of an alternative mechanism for setting extension and rotation of shanks of a self-retaining retractor, wherein the mechanism comprises a ball detent and associated grooves in accordance with some example embodiments of the disclosure.

FIG. 9 is an illustration of an alternative mechanism for setting extension and rotation of shanks of a self-retaining retractor, wherein the mechanism comprises a spring-loaded pull knob and associated grooves in accordance with some example embodiments of the disclosure.

FIG. 10 is an illustration of an alternative mechanism for setting extension and rotation of shanks of a self-retaining retractor, wherein the mechanism comprises a spring lock in accordance with some example embodiments of the disclosure.

FIG. 11 is an illustration of a self-retaining retractor that comprises molded portions in accordance with some example embodiments of the disclosure.

FIG. 12 is an illustration of a self-retaining retractor that comprises a shank that is recessed to receive an appendage during a surgical procedure in accordance with some example embodiments of the disclosure.

FIG. 13 is an illustration of a locking mechanism of a self-retaining retractor comprising a ratchet mechanism that comprises a leaf spring in accordance with some example embodiments of the disclosure.

FIG. 14 is an illustration of an aperture of a joint of a self-retaining retractor in accordance with some example embodiments of the disclosure.

FIGS. 15A, 15B and 15C (collectively FIG. 15) are illustrations of a self-retaining retractor comprising variations of a joint in accordance with some example embodiments of the disclosure.

FIG. 16 is an illustration of a self-retaining retractor that comprises overmolded portions in accordance with some example embodiments of the disclosure.

FIGS. 17A and 17B (collectively FIG. 17) are illustrations of self-retaining retractors comprising two horizontal shank sections that are straight and are disposed in respective receptacles of curved shank sections in accordance with some example embodiments of the disclosure.

FIGS. 18A and 18B (collectively FIG. 18) are illustrations of an arm member of a self-retaining retractor in accordance with some example embodiments of the disclosure.

FIG. 19 is an illustration of a joint between an arm member and a curved shank section of a self-retaining retractor in accordance with some example embodiments of the disclosure.

FIGS. 20A, 20B, 20C, 20D, 20E, 20F, 20G, 20H, 20I, 20J, 20K, 20L, 20M, 20N, 20O, 20P, 20Q, and 20R (collectively FIG. 20) are illustrations of arm members of a self-retaining retractor in accordance with some example embodiments of the disclosure.

FIGS. 21A and 21B (collectively FIG. 21) are illustrations of a self-retaining retractor in accordance with some example embodiments of the disclosure.

FIGS. 22A and 22B (collectively FIG. 22) are illustrations of a locking mechanism of a self-retaining retractor comprising a ratchet mechanism that comprises a metal leaf spring with a molded ratchet release in accordance with some example embodiments of the disclosure.

FIG. 23 is an illustration of a self-retaining retractor that comprises a third prong and associated arm member in accordance with some example embodiments of the disclosure.

Many aspects of the disclosure can be better understood with reference to these figures, which illustrate the aspects and subaspects of the disclosure described in the foregoing Summary section. The elements and features shown in the figures are not necessarily to scale, emphasis being placed upon clearly illustrating principles of example embodiments of the disclosure. Moreover, certain dimensions and features may be exaggerated to help visually convey such principles. In the figures, reference numerals often designate like or corresponding, but not necessarily identical, elements throughout the several views.

### DETAILED DESCRIPTION OF EXAMPLE EMBODIMENTS

The technology will be discussed more fully below with reference to the Figures, which provide additional information regarding representative or illustrative embodiments of the disclosure. The present technology can be embodied in many different forms and should not be construed as limited to the embodiments set forth herein; rather, these embodiments are provided so that this disclosure will be thorough and complete, and will fully convey the scope of the technology to those having ordinary skill in the art. Furthermore, all “examples,” “embodiments,” and “exemplary embodiments” provided herein are intended to be non-limiting and among others supported by representations of the disclosure.

Those of ordinary skill in the art having benefit of this disclosure will be able, without undue experimentation, to combine compatible elements and features that are described at various places in this written description, which includes text and illustrations. That is, the illustrations and specification are organized to facilitate practicing numerous combinations, such as by combining an element of one illustrated embodiment with another element of another illustrated embodiment or by combining a feature disclosed in an early paragraph of the specification with another element disclosed in a later paragraph of the specification.

This document includes sentences, paragraphs, and passages (some of which might be viewed as lists) disclosing alternative components, elements, features, functionalities, usages, operations, steps, etc. for various embodiments of the disclosure. Unless clearly stated otherwise, all such lists, sentences, paragraphs, passages, and other text are not exhaustive, are not limiting, are provided in the context of describing representative examples and variations, and are among others supported by various embodiments of the disclosure. Accordingly, those of ordinary skill in the art having benefit of this disclosure will appreciate that the disclosure is not constrained by any such lists, examples, or alternatives. Moreover, the inclusion of lists, examples, embodiments, and the like (where provided as deemed beneficial to the reader) may help guide those of ordinary skill in practicing many more implementations and instances that embody the technology without undue experimentation, all of which are intended to be within the scope of the claims.

This disclosure includes figures and discussion in which features and elements of certain embodiments may be organized into what might be characterized as functional units, blocks, subsystems, or modules. And, certain processes and methods may be organized into blocks or into steps. Such organization is intended to enhance readership and to facilitate teaching the reader about working principles of the technology and about making and using an abundance of embodiments of the disclosure. The organization is not intended to force any rigid divisions or partitions that would limit the disclosure. In practice, the flexibility of the technology and the depth of this disclosure supports dispersing or grouping functionalities, elements, and features in many different ways. The inclusion of an element or function in one block, unit, module, or subsystem verses another may be substantially arbitrary in many instances, with the divisions being soft and readily redrawn using ordinary skill in combination with the teaching provided herein. Accordingly, functional blocks, modules, subsystems, units, and the like can be combined, divided, repartitioned, redrawn, moved, reorganized, or otherwise altered without deviating from the scope and spirit of the disclosure. This is not to say that, nor will it support a conclusion that, any disclosed organizations and combinations are not novel, are not inventive, or are obvious.

Certain steps in the processes and methods disclosed or taught herein, may naturally need to precede others to achieve desirable functionality. However, the disclosure is not limited to the order of the described steps if such order or sequence does not adversely alter functionality to the extent of rendering the technology inoperable or nonsensical. That is, it is recognized that some steps of a process or method may be performed before or after other steps or in parallel with other steps without departing from the scope and spirit of the disclosure.

In some instances, a process or method (for example that entails using, making, or practicing) may be discussed with reference to a particular illustrated embodiment, application, or environment. For example, a process flow may reference or be discussed with reference to a figure. Those of skill in the art will appreciate that any such references are by example and are provided without limitation. Accordingly, the disclosed processes and methods can be practiced with other appropriate embodiments supported by the present disclosure and in other appropriate applications and environments. Moreover, one of ordinary skill in the art having benefit of this disclosure will be able to practice many variations of the disclosed methods and processes as may be appropriate for various applications and embodiments.

The term “couple,” as used herein, generally refers to joining, linking, connecting, or associating something with something else. When two things couple or something couples with something else, they may directly couple or indirectly couple via another, intervening component, element, or module. A first component may couple to or with a second component via the first component comprising the second component. Moreover, a first thing can couple to or with a second thing without physical contact, for example optically or via sound. The term can thus be read with a plain and ordinary meaning.

The term “coupled,” as used herein in a context of a first thing being coupled to or with a second thing, generally refers to the first thing being joined, linked, connected, or associated to or with the second thing. When something is coupled to or with something else, they may be directly coupled or indirectly coupled via another, intervening component, element, or module. Moreover, a first component may be coupled to or with a second component via the first component comprising the second component. A first thing can be coupled to or with a second thing without physical contact, for example optically or via sound. The term can thus be read with a plain and ordinary meaning.

The term “operably coupled,” as used herein in the context of two things being operably coupled, generally refers to the two things being coupled in a way that allows, supports, or facilitates something to work or operate. The term can thus be read with an ordinary and customary meaning.

When the terms “a” or “an” are used herein, one or more is to be generally understood, except when more than one would be nonsensical in context or would adversely alter functionality to the extent of rendering technology inoperable.

As one of ordinary skill in the art will appreciate, each of the terms “approximate” and “approximately,” as used herein, provides an industry-accepted tolerance for the associated term modified. Such industry-accepted tolerances range from less than one percent to ten percent and correspond to, but are not limited to, component values, dimensions, volumes, weights, signal levels, process variations, operational targets, and manufacturing tolerance.

The terms “substantial” and “substantially,” as used herein, are words of degree accommodating deviations that a skilled artisan would recognize as unintentional deviation from a target value or as inconsequential.

In each instance in which a number is disclosed for an embodiment, it is intended that approximately the number is disclosed for an embodiment and that substantially the number is disclosed for an embodiment. For example, if the specification discloses an embodiment comprising a dimension of 1.0 millimeter, it will be understood that a disclosed embodiment comprises a dimension of approximately 1.0 millimeter and that a disclosed embodiment comprises a dimension of substantially 1.0 millimeter.

In each instance in which a range of numbers is disclosed for an embodiment, it is intended that approximately the range of numbers is disclosed for an embodiment and that substantially the range of numbers is disclosed for an embodiment. For example if the specification discloses an embodiment comprising a dimension in a range of 1.0 to 2.0 millimeters, it will be understood that a disclosed embodiment comprises a dimension in an approximate range of 1.0 to 2.0 millimeters. Further, in this example, a disclosed embodiment comprises a dimension in a range of approximately 1.0 millimeter to approximately 2.0 millimeters. Further, in this example, a disclosed embodiment comprises a dimension in a substantial range of 1.0 to 2.0 millimeters. Further, in this example, a disclosed embodiment comprises a dimension in a range of substantially 1.0 millimeter to substantially 2.0 millimeters.

As will be appreciated by those of skill in the art, unless clearly specified otherwise, the values provided herein intend to reflect commercial design practices or nominal manufacturing targets. For example, what may be described or specified as having a dimension of one millimeter or 1.0 mm, may deviate from one millimeter or 1.0 mm when implemented in a commercial product due to fabrication error, warpage, or customary tolerances.

Turning now to the Figures, some illustrated embodiments will be further discussed.

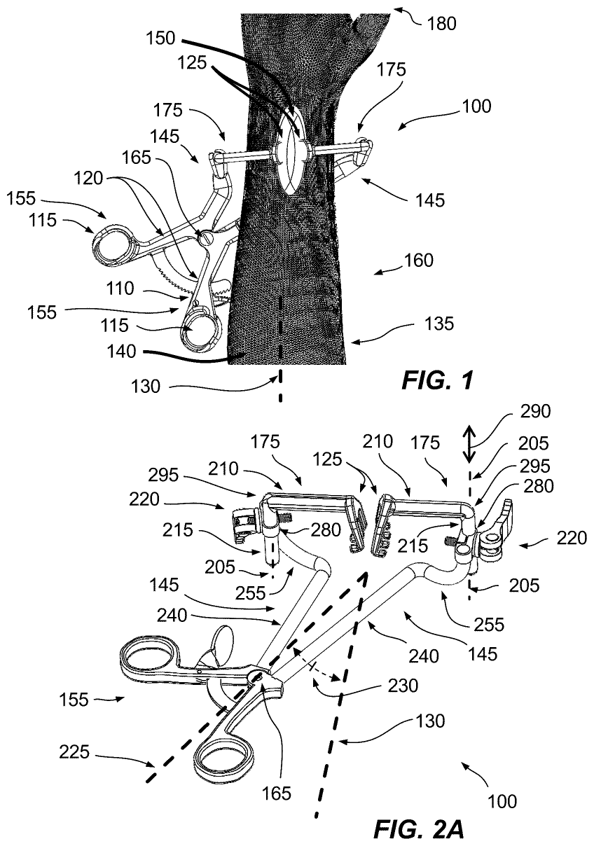

FIG. 1 illustrates a representative application of an example system for holding open a surgical incision 150, wherein an example self-retaining retractor 100 holds open the surgical incision 150 according to some embodiments of the disclosure. As illustrated in the example of FIG. 1, the surgical incision 150 is on a volar side 140 of a forearm 160 that comprises an appendage axis 130. In some other example embodiments, the surgical incision 150 can be on an upper arm, an elbow, a hand, a digit of a hand, a lower leg, a foot, an upper leg, a knee, a neck, or other appendage of a human or non-human animal undergoing a surgical procedure (not an exhaustive list).

In the illustrated example embodiment of FIG. 1, the self-retaining retractor 100 comprises a handle 155, which comprises pair of finger rings 115, that is disposed on a dorsal side 135 of the forearm 160, opposite the surgical incision 150. A pair of handle members 120 extend from the finger rings 115 to a pivot joint 165. A locking mechanism 110 extends between the handle members 120. A pair of prongs 125 extend into the surgical incision 150 and hold the incision 150 open. A pair of shanks 145 extend from the pivot joint 165 to the pair of prongs 125. Each shank 145 comprises a respective arm member 175. In some example embodiments, the arm member 175 can comprise or be viewed as a member comprising a boom, a jib, or an extension.

Between the pivot joint 165 and the prongs 125, the two shanks 145 extend along the appendage axis 130 and over the appendage axis 135. The shank 145 on the left extends helically clockwise as that shank 145 extends from the dorsal side 135 of the appendage axis 130 to the volar side 140 of the appendage axis 130. The shank 145 on the right extends helically counterclockwise as that shank 145 extends from the dorsal side 135 of the appendage axis 130 to the volar side 140 of the appendage axis 130. In the preceding two sentences, the clockwise and counterclockwise directions are taken from the arbitrary perspective of an observer positioned at an elbow (not illustrated) of the forearm 160 and looking distally (down the appendage axis 130 towards the hand 180).

In some example operations, a user actuates the self-retaining retractor by applying compressive force to the finger rings 115, such as by squeezing the finger rings 115 together so that the finger rings 115 move towards one another. The self-retaining retractor 100 translates the compressive force between the finger rings 115 into expansive force that the prongs 125 apply to the surgical incision 150 to open the surgical incision 150. The locking mechanism 110 maintains a user-defined separation between the finger rings 115, thereby maintaining a corresponding user-defined separation between the prongs 125 and thereby holding the surgical incision 150 open. In some example embodiments, the self-retaining retractor 100 is concurrently entrapped to, or otherwise constrained by, the forearm 160, so that the self-retaining retractor 100 can be held in place without requiring ongoing assistance from the user. A surgeon can thus readily conduct a surgical procedure within the open surgical incision 150, for example installing a bone plate or other orthopedic implant, repairing a nerve, removing a tumor, or otherwise operating on tissue accessed through the surgical incision 150. With the self-retaining retractor 100 retaining its position on the forearm 160, the surgeon's hands can be free to perform the surgical procedure.

In the example embodiment of FIG. 1, the handle is biased to a lateral side of the forearm. The illustrated biasing of the handle can facilitate x-raying the forearm during surgery with fidelity, by moving the handle out of or to a peripheral area of the x-ray field of view. Furthermore, in some surgical procedures many x-rays may be taken during the surgery to confirm placement of an implant. With the self-retaining retractor 100 composed of radiolucent materials, the surgeon can save significant time and reduce potential injury to the patient by not having to remove the self-retaining retractor 100 to perform the imaging. In some surgeries, conventional retractors may be removed as many as 30 times and then replaced before an adequate positioning of an implant is accomplished. The illustrated biasing of the handle 155 can further impede pronation of the forearm 160 during surgery or other tendency of an appendage to rotate. In some example embodiments, the handle 155 is biased so as to be disposed between the forearm 160 and an abdomen of a patient and impedes pronation via interaction with a solid surface (not illustrated) that supports the forearm during surgery, for instance a surgical table.

In some example embodiments, the self-retaining retractor 100 is composed of stainless steel, titanium, aluminum, plastic, polymer, a known medical alloy, or a combination thereof (not an exhaustive list).

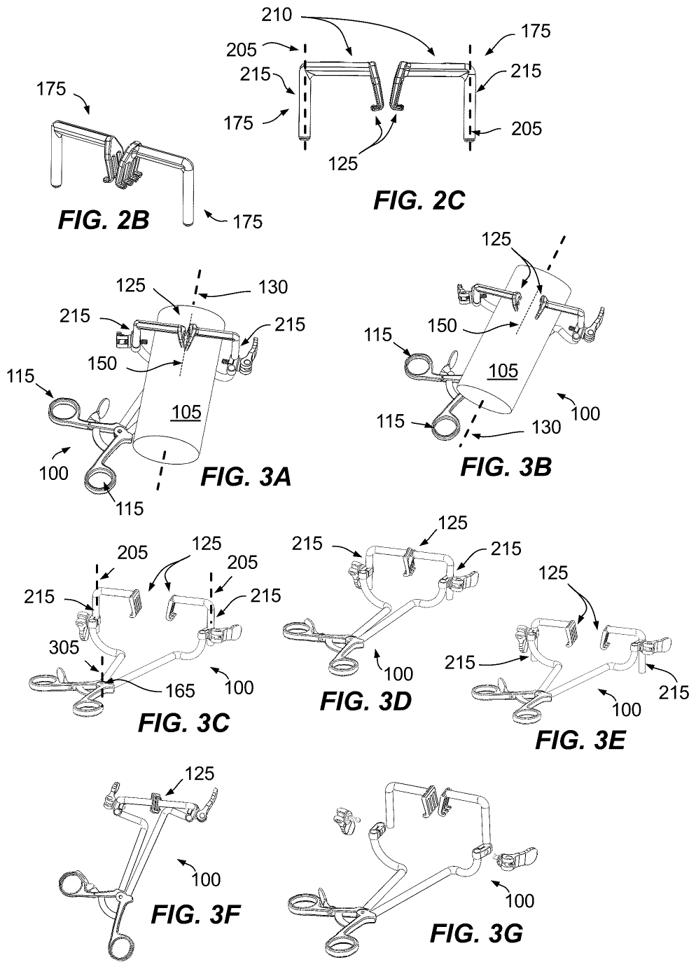

Turning now to FIGS. 2A, 2B, and 2C, these figures illustrate an example self-retaining retractor 100 according to some embodiments of the disclosure. In some example embodiments, FIGS. 2A, 2B, and 2C illustrate further details of the self-retaining retractor 100 of FIG. 1, and the following paragraphs will refer to FIGS. 2A, 2B, and 2C as such without limitation.

FIG. 2A illustrates a full view of the self-retaining retractor 100, for example as fully assembled. FIGS. 2B and 2C illustrate two views of example embodiments of the arm members 175 of the self-retaining retractor 100 that are vertically and rotationally adjustable as further discussed below.

In the example embodiment of FIG. 2, each shank 145 comprises a respective horizontal shank section 240. The two horizontal shank sections 240 have different lengths. The two different lengths bias the handle 155 of the self-retaining retractor 100 laterally relative to the forearm 160 (see FIG. 1) or other appendage. Thus, the self-retaining retractor 100 has a retractor axis 225 that is skewed or angled relative to the appendage axis 130. In the illustrated example of FIG. 2A, a resulting obtuse angle 230 that is in a range of 20 degrees to 50 degrees offsets the retractor axis 225 from the appendage axis 130. Other embodiments may have angular offset 230 outside this range, for example below 20 degrees or greater than 50 degrees.

Each shank 145 further comprises a curved shank section 255 that extends from the horizontal shank section 240 to a respective joint 280 that has an associated lock 220. In the illustrated example embodiment of FIG. 2, each lock 220 comprises a cam lever lock. In some example embodiments, the curved shank section 255 is curved according to curvature of an appendage, such as the forearm 160 illustrated in FIG. 1. Thus, the curved shank section 255 can conform to or follow along surface contours of the forearm 160 or another appendage that comprises the surgical incision 150 that is to be held open.

The arm member 175 of each shank 145 comprises a vertical shank section 215 and a transverse shank section 210. The vertical shank section 215 extends vertically from the joint 280 along a respective vertical axis 205. The transverse shank section 210 extends from the vertical shank section 215 to a respective prong 125. As illustrated, each arm member 175 comprises a rigid abrupt bend 295 between the vertical shank section 215 and the transverse shank section 210. In some example embodiments (not illustrated by FIG. 2), another joint exists between the vertical shank section 215 and the transverse shank section 210, thereby providing at least one additional degree of freedom relative to the embodiment of FIG. 2.

As further discussed below with reference to more figures, the illustrated vertical shank sections 215 of the arm members 175 extend through the joints 280 to provide an adjustable height 290. The user can thus vary extension of each of the prongs 125 into the surgical incision 150 and use the lock 220 to set the prongs 125 to a desired amount of extension.

The adjustable height 290 can further facilitate securing the self-retaining retractor 100 to the forearm 160. The horizontal shank sections 240 can be sandwiched between the forearm 160 and a flat surface, such as an operating table, that supports the forearm 160 during surgery. So sandwiched, the self-retaining retractor 100 can be securely positioned against the dorsal side 135 of the forearm 160. From this stable position, the arm member 175 can be set to a desired position so the prongs 125 desirably hold the surgical incision 150 open. The fixed, stable positioning of the self-retaining retractor 100 relative to the forearm 160 further helps prevent the prongs from sliding or otherwise inadvertently moving relative to the surgical incision 150, thereby eliminating, avoiding, or reducing any need to repeatedly reposition the self-retaining retractor 100 during a surgical procedure. The resulting stability thus helps avoid disengagement between the prongs 125 and tissue of the surgical incision 150.

In the illustrated embodiment of FIGS. 1, 2A, 2B, and 2C, each vertical shank section 215 can rotate in the joint 280 about its associated vertical axis 205. Thus, each prong 125 can rotate about its associated vertical axis 205. Once the user establishes a desired rotation and height for each prong 125, the user can lock the vertical shank sections 215 in place. The user can accordingly set prong depth into the surgical incision 150 and prong rotational angle. This capability can reduce unwanted damage to tissue structures adjacent the surgical incision 150, as well as avoiding unintentional displacement of the self-retaining retractor 100 as described above.

Turning now to FIGS. 3A, 3B, 3C, 3D, 3E, 3F, and 3G, these figures illustrate an example self-retaining retractor 100 in combination with a cylinder 105 that represents an appendage comprising a surgical incision 150 that the self-retaining retractor 100 is configured to hold open according to some example embodiments of the disclosure. In some example embodiments, FIG. 3 illustrates further details of the self-retaining retractor 100 of FIGS. 1 and 2, and the following paragraphs will refer to FIGS. 3A, 3B, 3C, 3D, 3E, 3F, and 3G, as such without limitation.

FIGS. 3A and 3B illustrate the self-retaining retractor 100 and the associated cylinder 105. In FIG. 3A, the finger rings 115 are open and the prongs 125 are situated proximate to each other. In FIG. 3B, the finger rings 115 have been pressed together, resulting in separation of the prongs 125.

FIGS. 3C, 3D, and 3E illustrate the vertical shank sections 215 set in three respective positions. In FIG. 3C, the vertical shank sections 215 are locked to provide large separation between the prongs 125 and the appendage axis 105, for example to accommodate a large-diameter forearm 160. In FIG. 3D, the vertical shank sections 215 are locked to provide intermediate separation between the prongs 125 and the appendage axis 130, for example to accommodate an intermediate-diameter forearm 160. In FIG. 3E, the vertical shank section 215 are locked to provide small separation between the prongs 125 and the appendage axis 130, for example to accommodate an small-diameter forearm 160. This adjustability facilitates the self-retaining retractor 100 securely holding onto the forearm 160 or other appendance, thereby avoiding frequent, unwanted dislodgement of the prongs 125 with the surgical incision 150.

FIG. 3F corresponds to FIG. 3A and illustrates the self-retaining retractor with the finger rings 115 open and the prongs 125 closed. FIG. 3G illustrates an exploded view of the self-retaining retractor 100.

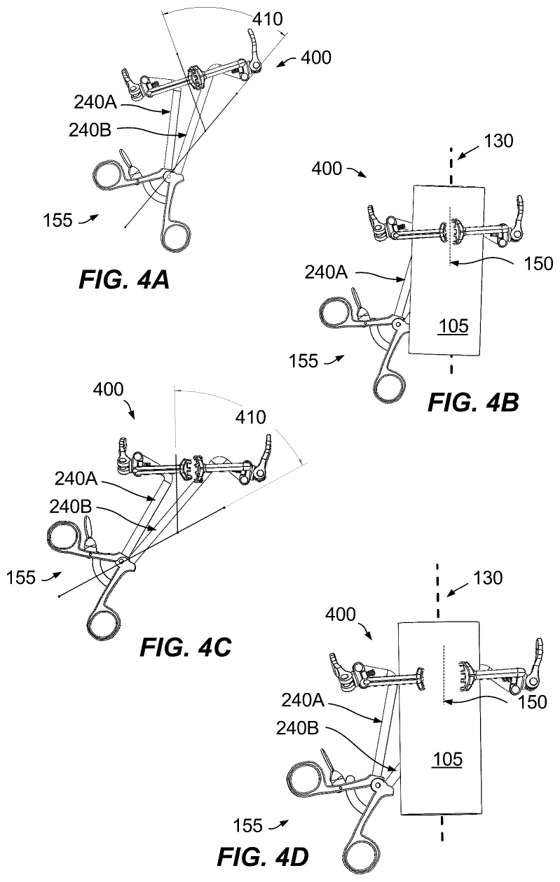

Turning now to FIGS. 4A, 4B, 4C, and 4D, these figures illustrate an example self-retaining retractor 400 that comprises different shank lengths in combination with a cylinder 105 that represents an appendage comprising a surgical incision 150 that the self-retaining retractor 400 is configured to hold open according to some embodiments of the disclosure. These figures illustrate different retractor configurations that can result from varying lengths of the horizontal shank sections 240A, 240B. Varying length of the horizontal shank section 240A relative to the horizontal shank section 240B can position the handle 155 of the self-retaining retractor 400 more oblique to the central axis 130 of the appendage 105, assisting in customizing a level of pronation reduction that may be helpful.

As illustrated by FIGS. 4A and 4B, changing the relative lengths of the horizontal shank sections 240A, 240B affects the angle 410. The horizontal shank section 240B of the configuration that FIG. 4C illustrates is longer than the horizontal shank section 240 B of the configuration that FIG. 4A illustrates. Consequently, the angle 410 of the configuration that FIG. 4C illustrates is larger than the angle 410 of the configuration that FIG. 4A illustrates. FIGS. 4A and 4B thus illustrate how lengthening the horizontal shank section 240B can increase the angle 410.

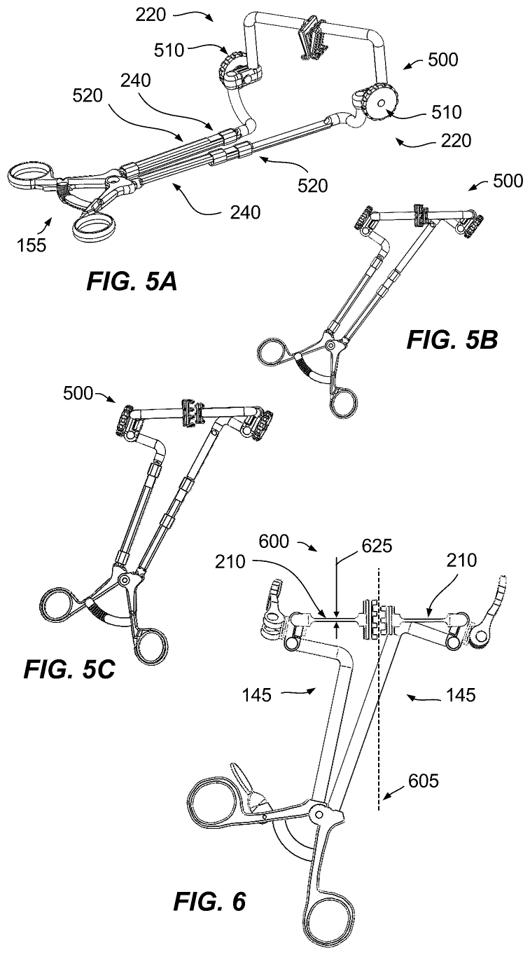

Turning now to FIGS. 5A, 5B, and 5C, these figures illustrate an example self-retaining retractor 500 that comprises adjustable shank lengths according to some embodiments of the disclosure. Additionally, in the embodiment that FIG. 5 illustrates, the lock 220 comprises a knob 510 that locks via turning, as an alternative to the cam lever lock that FIGS. 1, 2, and 3 illustrate as discussed above.

As illustrated in FIGS. 5A, 5B, and 5C, the length of each of the horizontal shank sections 240 is user configurable. In the illustrated example, each horizontal shank section 240 comprises a telescoping locking mechanism 520. In some example embodiments, the telescoping locking mechanisms 520 comprise collets or twist locks. In some example embodiments, each telescoping locking mechanism 520 comprises a lever lock, flip lock, button lock, collar lock, or other suitable means for locking each horizontal shank section 240 to a user-defined length. In some example embodiments, each telescoping locking mechanism 520 comprises a spline joint or other suitable means for impeding rotation at the mechanism 520.

The user can thus set lengths of the horizontal shank sections 240 to provide a desired level of lateral offset as discussed above with reference to FIG. 4, among other places. Further, the user can set the lengths of the horizontal shank sections 240 so that the handle 155 is biased to either lateral side of an appendage, such as the forearm 160 that FIG. 1 illustrates. For instance, the handle 155 can be oriented either towards or away from a patient torso. Moreover, a user can bias the handle 155 to one lateral side for impeding pronation of a left forearm that is undergoing surgery and can biased the handle 155 to the opposite lateral side for impeding pronation of a right forearm that is undergoing surgery.

Turning now to FIG. 6, this figure illustrates an example self-retaining retractor 600 that comprises a pair of shanks 145, respective portions of which are compliant, according to some embodiments of the disclosure. In some example embodiments, a reference plane 605 can extend between the prongs 125 of the self-retaining retractor 600. The reference plane can comprise the appendage axis (illustrated in FIG. 3A and in other figures). The transverse shank sections 210 can be compliant to the reference plane 605 in some example embodiments.

In the illustrated example embodiment, the transverse shank section 210 comprises an elongate cross section. In some example embodiments, the elongate cross section comprises a height (extending into the page in the view of FIG. 6) and a width 625. In some example embodiments, the height is in a range of approximately two to approximately five times the width 625. In some example embodiments, at least a longitudinal portion of the transverse shank section 210 comprises an elastomer or a polymer. In some example embodiments, a longitudinal portion of the transverse shank section 210 comprises at least one of acetal, polyamide, silicone, or other appropriate polymeric material.

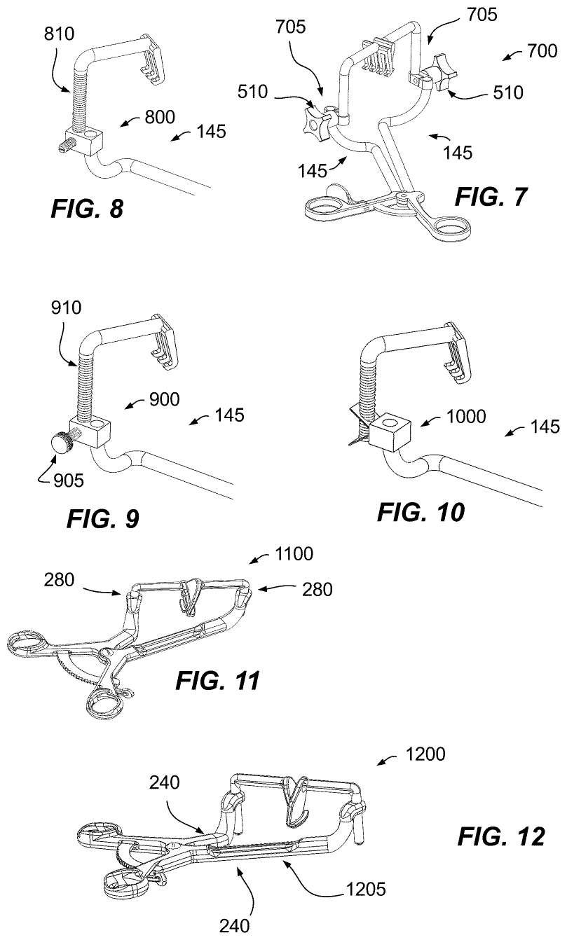

Turning now to FIG. 7, this figure illustrates an example self-retaining retractor 700 that comprises a pair of shanks 145, respective portions of which are extendable and rotatable, each with an example mechanism 705 for setting extension and rotation that comprises an example knob 510 according to some embodiments of the disclosure.

Turning now to FIG. 8, this figure illustrates another example mechanism 800 for setting extension and rotation of shanks 145 of an example self-retaining retractor, wherein the mechanism 800 comprises a ball detent and associated grooves 810 according to some embodiments of the disclosure. The mechanism 800 can comprise an embodiment of a lock 220 and a joint 280 as illustrated in FIG. 2A and discussed above.

Turning now to FIG. 9, this figure illustrates another example mechanism 900 for setting extension and rotation of shanks 145 of an example self-retaining retractor, wherein the mechanism 900 comprises a spring-loaded pull knob 905 and associated grooves 910 according to some embodiments of the disclosure. The mechanism 900 can comprise an embodiment of a lock 220 and a joint 280 as illustrated in FIG. 2A and discussed above.

Turning now to FIG. 10, this figure illustrates another example mechanism 1000 for setting extension and rotation of shanks 145 of an example self-retaining retractor, wherein the mechanism 1000 comprises a spring lock according to some embodiments of the disclosure. The mechanism 1000 can comprise an embodiment of a lock 220 and a joint 280 as illustrated in FIG. 2A and discussed above.

Embodiments of the lock 220 and joint 280 as illustrated in FIG. 2A can comprise other suitable locking and joining mechanisms. In some example embodiments, a suitable locking and joining mechanism can comprise a pin lock interface. In some example embodiments, a suitable locking and joining mechanism can comprise a spring-loaded ball detent. In some example embodiments, a suitable locking and joining mechanism can comprise a keyed joint.

Turning now to FIG. 11, this figure illustrates an example self-retaining retractor 1100 that comprises molded portions according to some embodiments of the disclosure. In some example embodiments, at least a portion of the self-retaining retractor 110 comprises polyamide, polyether ether ketone, acetal, polycarbonate, polyetherimide, or other appropriate polymer. In some example embodiments, the joints 280 comprise a molded polymer and can further comprise a friction fit that supports free rotation about the vertical axis 205 (see FIG. 2A) while holding vertical position. Such free rotation can allow the device to reposition relative to the patient's appendage, thereby reducing localized tissue stress, limiting torsional loads applied to the incision site, and protecting surrounding soft tissue from unnecessary pressure or shear during surgical manipulation.

Turning now to FIG. 12, this figure illustrates an example self-retaining retractor 1200 that comprises a horizontal shank section 240 that is recessed to receive an appendage during a surgical procedure according to some embodiments of the disclosure. In example usage, the user positions the forearm 160 (see FIG. 1) in the recess 1205, with the horizontal shank section 240 sandwiched between the forearm 160 and an operating table or other stable surface.

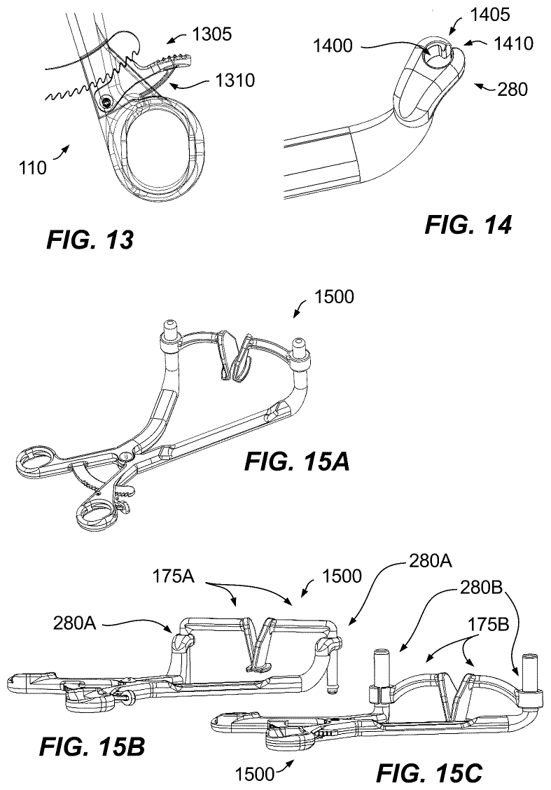

Turning now to FIG. 13, this figure illustrates an example locking mechanism 110 of a self-retaining retractor comprising a ratchet mechanism 1305 that comprises a leaf spring 1310 in accordance with some example embodiments of the disclosure.

Turning now to FIG. 14, this figure illustrates an example aperture 1400 of an example joint 280 of a self-retaining retractor in accordance with some example embodiments of the disclosure. The aperture 1400 can be sized to receive the vertical shank section 215 (see FIG. 2A). In the illustrated example embodiment of FIG. 14, the aperture 1400 comprises a slit 1410 and associated protrusion 1405 for providing vertical support and a friction fit. The slit 1410 can provide spring loading in some example embodiments. In some example embodiments, a ratio of aperture length to diameter of the vertical shank section 215 is less than or equal to 1.5, resulting in binding without undue axial motion.

Turning now to FIGS. 15A, 15B and 15C, these figures illustrate example self-retaining retractors 1500 comprising variations of a joint 280A, 280B in accordance with some example embodiments of the disclosure. In the example embodiment of FIG. 15B, the joint 280A comprises a male arm member 175A. In the example embodiment of FIG. 13C, the joint 280B comprises a female arm member 175B.

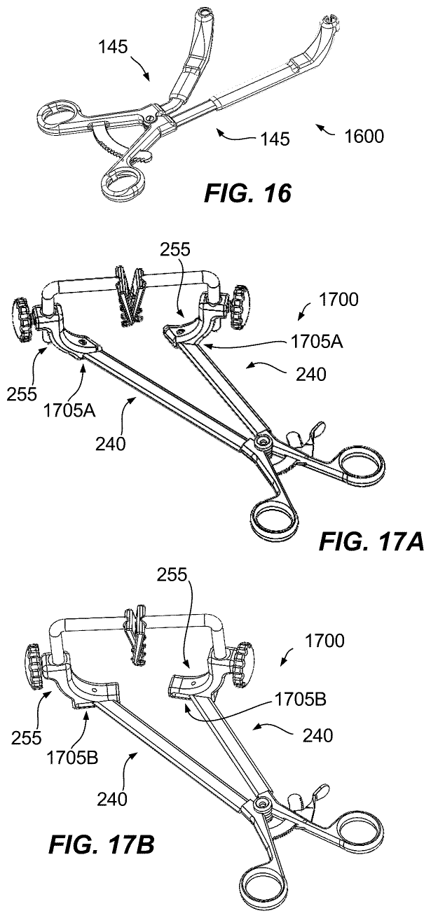

Turning now to FIG. 16, this figure illustrates an example self-retaining retractor 1600 that comprises example overmolded portions according to some embodiments of the disclosure. In some example embodiments, at least a portion of at least one of the shanks 145 is overmolded to suppress bending and/or torsion. In some example embodiments, at least one of the shanks 145 comprises a metallic core that is overmolded in a suitable polymer, for example acetal, polyamide, silicone, polycarbonate polyetherimide, polyether ether ketone, or other appropriate polymeric material.

Turning now to FIGS. 17A and 17B, these figures illustrate example self-retaining retractors 1700 comprising two example horizontal shank sections 240 that are straight and are disposed in respective example receptacles 1705A, 1750B of example curved shank sections 255 according to some embodiments of the disclosure. The receptacles 1750B that FIG. 17B illustrates provide adjustment between the horizontal shank sections 240 and the associated curved shank sections 255.

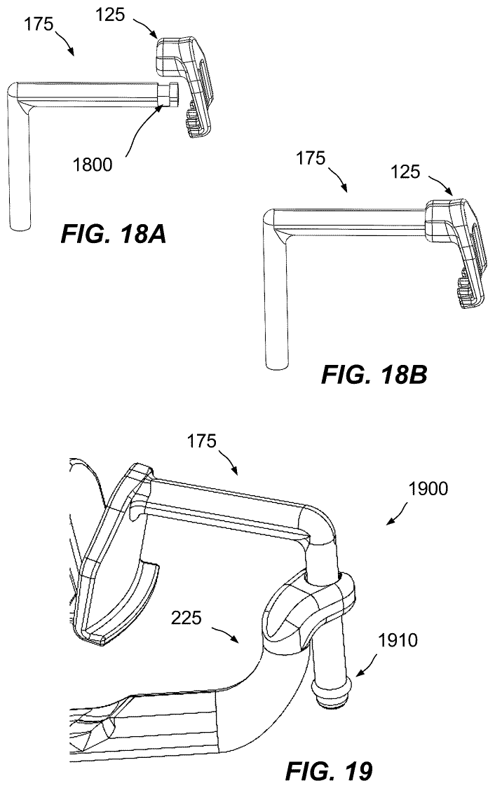

Turning now to FIGS. 18A and 18B, these figures illustrate an example arm member 175 of an example self-retaining retractor according to some embodiments of the disclosure. As illustrated, the arm member 175 comprises two parts, wherein the tine 125 comprises a removable end effector that is received in a groove or recess 1800.

Turning now to FIG. 19, this figure illustrates an example joint 1900 between an example arm member 175 and an example curved shank section 255 of a self-retaining retractor according to some embodiments of the disclosure. In the illustrated embodiment, the joint comprises a snap-in coupling with a rib 1910 that prevents lifting of the arm member 175 under load.

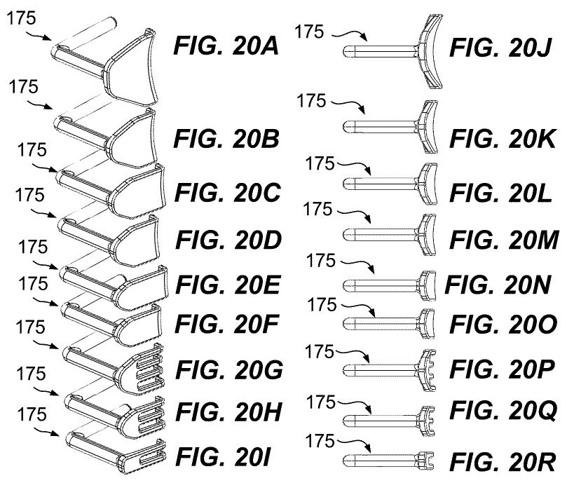

Turning now to FIGS. 20A, 20B, 20C, 20D, 20E, 20F, 20G, 20H, 20I, 20J, 20K, 20L, 20M, 20N, 20O, 20P, 20Q, and 20R, these figures illustrate example arm members 175 of a self-retaining retractor according to some embodiments of the disclosure. In the illustrated example embodiments (which are not exhaustive), the variations are suited to various applications or personal preferences of a surgeon or other user. Curved profiles can distribute load to tissue in some embodiments. In some embodiments, the geometry provides a relatively open view to the surgeon or other user.

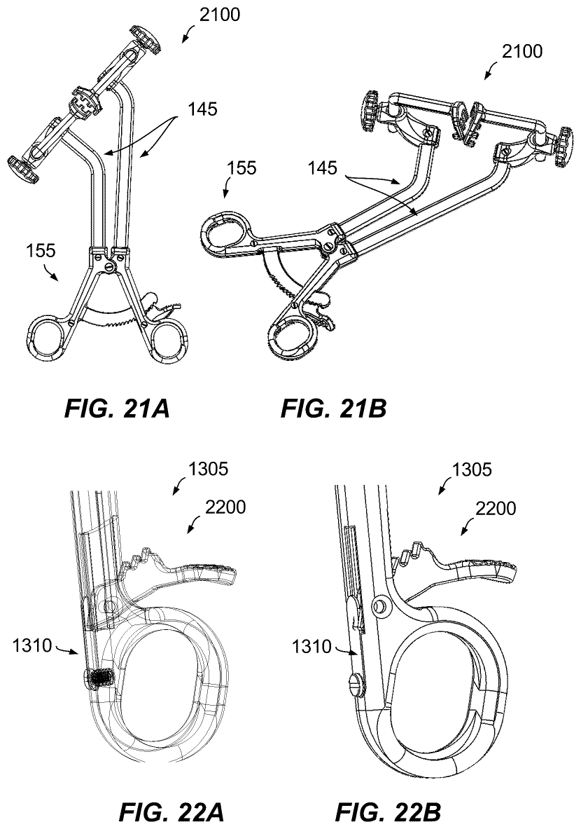

Turning now to FIG. 21, this figure illustrates an example self-retaining retractor 2100 according to some embodiments of the disclosure. In the example embodiment of FIG. 21, the self-retaining retractor 2100 can comprise metallic shanks 145 with a molded handle 155.

Turning now to FIGS. 22A and 22B, these figures illustrate an example locking mechanism 110 of a self-retaining retractor comprising a ratchet mechanism 1305 that comprises a metal leaf spring 1310 with a molded ratchet release 2200 in accordance with some example embodiments of the disclosure.

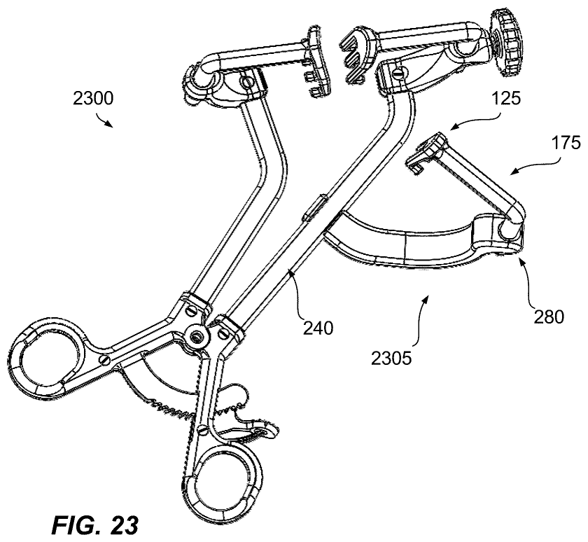

Turning now to FIG. 23, this figure illustrates an example self-retaining retractor 2300 that comprises a third example prong 125 and associated example arm member 175 in accordance with some example embodiments of the disclosure. The prong 125 and arm member 175 comprise an arm 2305 that is releasably attachable to the horizontal shank section 240. The arm 2305 further comprises a third joint 280 for a surgeon or other user to set the third prong 125 to a desired configuration as deemed beneficial for a particular surgery or other medical procedure. Accordingly, the user can deploy and configure the third prong 125 on an as-needed basis to provide retraction from an additional direction or vector.

Useful technology for holding open a surgical incision has been described. From the description, it will be appreciated that an embodiment of the disclosure overcomes limitations of the prior art. Those skilled in the art will appreciate that the technology is not limited to any specifically discussed application or implementation and that the embodiments described herein are illustrative and not restrictive. Furthermore, the particular features, structures, or characteristics that are set forth may be combined in any suitable manner in one or more embodiments based on this disclosure and ordinary skill. Those of ordinary skill having benefit of this disclosure can make, use, and practice a wide range of embodiments via combining the disclosed features and elements in many permutations without undue experimentation and further by combining the disclosed features and elements with what is well known in the art. This disclosure not only includes the illustrated and described embodiments, but also provides a roadmap for additional embodiments using the various disclosed technologies, elements, features, their equivalents, and what is well known in the art. From the description of the example embodiments, equivalents of the elements shown herein will suggest themselves to those skilled in the art, and ways of constructing other embodiments will appear to practitioners of the art. Therefore, the scope of the technology is to be limited only by the appended claims.

What is claimed is:

1. A method for holding open a surgical incision on a volar side of a forearm, the method comprising:
by a handle of a retractor disposed on a dorsal side of a forearm, receiving compressive force exerted on the handle by a user; and
by a pair of members and a pivot joint of the retractor that are operably coupled to the handle, converting the compressive force into expansive force between two prongs of the retractor disposed in the surgical incision on the volar side of the forearm, whereby the two prongs hold open the surgical incision.

2. The method of claim 1,
wherein the two prongs disposed in the surgical incision comprise:
a first prong disposed in the surgical incision and applying a first force to a first side of the surgical incision; and
a second prong disposed in the surgical incision applying a second force to a second side of the surgical incision, and
wherein the first force and the second force are substantially parallel or substantially collinear and oppose one another.

3. The method of claim 1,
wherein the two prongs disposed in the surgical incision comprise:
a first prong disposed in the surgical incision against a first side of the surgical incision; and
a second prong disposed in the surgical incision against a second side of the surgical incision,
wherein the first prong applies to the first side of the surgical incision a first force having a first magnitude and a first direction,
wherein the second prong applies to the second side of the surgical incision a second force having a second magnitude and a second direction,
wherein the first and second magnitudes are substantially equal, and
wherein the second direction is substantially opposite the first direction.

4. The method of claim 1, wherein the method further comprises
by the handle of the retractor disposed on the dorsal side of the forearm, pressing against a surface that supports the forearm during surgery to impede pronation of the forearm; and
by the self-retaining retractor, lengthening the first prong relative to the second prong responsive to user input.

5. The method of claim 1, wherein the handle of the retractor disposed on the dorsal side of the forearm is biased laterally relative to the forearm.

6. A retractor comprising:
a first member comprising:
a first finger ring;
a first portion of a pivot joint;
a first handle portion extending between the first finger ring and the pivot joint;
a first prong; and
a first shank extending between the pivot joint and the first prong;
a second member comprising:
a second finger ring;
a second portion of the pivot joint;
a second handle portion extending between the second finger ring and the pivot joint;
a second prong; and
a second shank extending between the pivot joint and the second prong;
the pivot joint; and
a locking mechanism extending between the first and second handle portions,
wherein the first shank:
extends lengthwise away from the pivot joint along a first side of an axis and then extends in a first rotational direction partially about the axis to a second side of the axis that opposes the first side of the axis; and
orients the first prong on the second side of the axis with the first prong extending towards the axis, and
wherein the second shank:
extends lengthwise away from the pivot joint along the first side of the axis and then extends in a second rotational direction partially about the axis to the second side of the axis, wherein the first and second rotational directions oppose one another; and
orients the second prong on the second side of the axis with the second prong extending towards the axis.

7. The retractor of claim 6, wherein the first prong curves in the first rotational direction as the first prong extends towards the axis,
wherein the second prong curves in the second rotational direction as the second prong extends towards the axis,
wherein the axis comprises an axis of an appendage, and
wherein the retractor is configured to hold open a surgical incision on the appendage.

8. The retractor of claim 6, wherein the first and second finger rings form a handle that is laterally offset relative to the axis.

9. The retractor of claim 6, wherein at least one of the first shank and the second shank comprises a straight section that is extendable.

10. The retractor of claim 6, wherein the first prong is rotatable about a second axis that is substantially perpendicular to the axis, and
wherein the second prong is rotatable about a third axis that is substantially perpendicular to the axis.

11. The retractor of claim 10, wherein the first shank comprises a first lock for fixing the first prong in a first rotational position about the second axis, and
wherein the second shank comprises a second lock for fixing the second prong in a second rotational position about the third axis.

12. The retractor of claim 6, wherein the first shank comprises a first section that extends in the first rotational direction partially about the axis to the second side of the axis,
wherein the second shank comprises a second section that extends in the second rotational direction partially about the axis to the second side of the axis,
wherein a first portion of the first section is extendable for setting first separation between the first prong and the axis, and
wherein a second portion of the second section is extendable for setting second separation between the second prong and the axis.

13. The retractor of claim 6, wherein a reference plane extends between the first prong and the second prong and comprises the axis,
wherein a first portion of the first shank is disposed adjacent the first prong and is compliant to the reference plane, and
wherein a second portion of the second shank is disposed adjacent the second prong and is compliant to the reference plane.

14. A method for holding open a surgical incision on a first side of an appendage, the method comprising:
providing a self-retaining retractor that comprises:
a handle comprising a pair of finger rings;
a pair of prongs disposed opposite the handle;
a joint disposed between the handle and the pair of prongs; and
a pair of members that extend between handle and the pair of prongs and that meet at the joint; and
by the self-retaining retractor, with the handle disposed adjacent to a second side of the appendage that is opposite the first side of the appendage, holding open the surgical incision.

15. The method of claim 14, wherein holding open the surgical incision comprises the pair of prongs extending into and holding open the surgical incision while each prong in the pair of prongs is free to rotate about a respective axis that extends vertically.

16. The method of claim 15, further comprising
by the self-retaining retractor, locking each prong in the pair of prongs to a user-selected angular position about the respective axis that extends vertically.

17. The method of claim 16, further comprising
by the self-retaining retractor, locking each prong in the pair of prongs to a respective user-selected extension length that sets a respective depth of extension into the surgical incision.

18. The method of claim 14, further comprising
by the self-retaining retractor, with the handle disposed between the second side of the appendage and a surface supporting the appendage during surgery, impeding rotation of the appendage.

19. The method of claim 14, further comprising
by the self-retaining retractor, with the handle biased laterally relative to the appendage and disposed between the second side of the appendage and a surface supporting the appendage, impeding rotation of the appendage via interaction between the biased handle and the surface.

20. The method of claim 14, further comprising
by the self-retaining retractor, with the handle disposed adjacent the second surface of the appendage and biased to a lateral side of the appendage, holding open the surgical incision on the first side of the appendage with the pair of prongs extending into the surgical incision.
